(19) United States
(12) Patent Application Publication
(10) Pub. No.: US 2024/0352421 A1
(43) Pub. Date: Oct. 24, 2024

(54) METHOD FOR IN-VITRO PRODUCTION OF A COHESIVE CARTILAGE CONSTRUCT

(71) Applicant: Chondro Engineering AS, Tromsø (NO)

(72) Inventor: Oddmund Johannes Johansen, Tomasjord (NO)

(73) Assignee: Chondro Engineering AS, Tromsø (NO)

(21) Appl. No.: 17/753,946

(22) PCT Filed: Sep. 18, 2020
(86) PCT No.: PCT/EP2020/076129

§ 371 Date: Mar. 18, 2022

(30) Foreign Application Priority Data
Sep. 20, 2019 (NO) 20191140

(51) Int. Cl. C12N 5/077; A61L 27/38

(52) U.S. Cl. CPC C12N 5/0655; A61L 27/3852; A61L 27/3895; C12N 2500/02; C12N 2513/00; C12N 2527/00

(57) ABSTRACT

A method for in-vitro production of a cohesive cartilage construct includes the following steps: a) propagating chondrogenic cells derived from a subject to allow formation of one or more cartilage micro constructs; b) putting a plurality of the cartilage micro constructs in motion to facilitate contact between the cartilage micro constructs and thereby allow formation of one or more fused cartilage micro constructs; and c) subjecting one or more of the fused cartilage micro constructs to mechanical stimulation in a hypoxic environment to allow formation of a cohesive cartilage construct.

### FIELD OF THE INVENTION

The present invention is in the field of medical engineering, and in particular cartilage tissue engineering for repair of cartilage lesions in clinical applications. The present invention provides a transplantable cohesive cartilage construct and a method for its in-vitro production. Further, the invention is directed to use of the transplantable cohesive cartilage construct in a surgical method for repairing damaged cartilage.

### BACKGROUND OF THE INVENTION

Human joint surfaces are covered by articular cartilage, a low friction, durable material that distributes mechanical forces and protects the underlying bone. Injuries to articular cartilage are common, especially in the knee. Such injuries occur most commonly in young active people and result in pain, swelling, and loss of joint motion. Damaged articular cartilage does not heal. Typically, degeneration of the surrounding uninjured cartilage occurs over time resulting in chronic pain and disability. Cartilage injuries therefore frequently lead to significant loss of productive work years and have enormous impact on patients' recreation and lifestyle.

Joint surface injuries may be limited to the cartilage layer or may extend into the subchondral bone. Cartilage injuries which do not penetrate the subchondral bone have limited capacity for healing (A. I. Caplan, Nippon Seikcigcka Gakkai Zasshi 63, 692-9 (1989)). This is due to properties inherent to the tissue. Nearly 95 percent of articular cartilage is extracellular matrix (ECM) that is produced and maintained by the chondrocytes dispersed throughout it. The ECM provides the mechanical integrity of the tissue. The limited number of chondrocytes in the surrounding tissue are unable to replace ECM lost to trauma. A brief overproduction of matrix components by local chondrocytes has been observed (R. G. Johnson, A. R. Poole, Exp. Pathol. 38, 37-52 (1990)); however, the response is inadequate for the repair of clinically relevant defects. Cellular migration from the vascular system does not occur with pure chondral injury and extrinsic repair is clinically insignificant.

Osteochondral injuries, in which the subchondral bone plate is penetrated, can undergo healing due to the influx of reparative cells from the bone marrow (A. I. Caplan, Nippon Seikeigeka Gakkai Zasshi 63, 692-9 (1989)). Numerous studies have shown, however, that the complex molecular arrangement of the ECM necessary for normal cartilage function is not recapitulated. The repair response is characterized by formation of fibrocartilage, a mixture of hyaline cartilage and fibrous tissue. Fibrocartilage lacks the durability of articular cartilage and eventually undergoes degradation during normal joint use. Many osteochondral injuries become clinically asymptomatic for a period of a few to several years before secondary degeneration occurs. However, like isolated chondral injuries, these injuries ultimately result in poor joint function, pain, and disability.

Current methods of surgical restoration of articular cartilage basically fall into three categories: 1) stimulation of fibrocartilaginous repair; 2) osteochondral grafting; and 3) autologous chondrocyte transplantation.

Fibrocartilage, despite its relatively poor mechanical properties, can provide temporary symptomatic relief in articular injuries. Several surgical techniques have been developed to promote the formation of fibrocartilage in areas of cartilage damage. These include subchondral drilling, abrasion, and microfracture. The concept of these procedures is that penetration of the subchondral bone allows chondroprogenitor cells from the marrow to migrate into the defect and effect repair.

In osteochondral grafting, articular cartilage is harvested with a layer of subchondral bone and transplanted into the articular defect. Fixation of the graft to the host is accomplished through healing of the graft bone to the host bone. The major advantage of this technique is that the transplanted cartilage has the mechanical properties of normal articular cartilage and therefore can withstand cyclical loading. The major disadvantages are donor-site morbidity (in the case of autograft) and risk of disease transmission (in the case of allograft). Additionally, tissue rejection can occur with allografts which compromises the surgical result.

Autologous chondrocyte transplantation is a method of cartilage repair that uses isolated chondrocytes. Clinically, this is a two-step treatment in which a cartilage biopsy is first obtained and then, after a period of ex-vivo processing, cultured chondrocytes are introduced into the defect (D. A. Grande, M. I. Pitman, L. Petersen, D. Menche, M. Klein, Journal of Orthopaedic Research 7, 208-218 (1989)). During the ex-vivo processing, the ECM is removed, and the chondrocytes are cultured under conditions that promote cell division. Once a suitable number of cells are produced, they are transplanted into the articular defect. Containment is typically provided by a patch of periosteum which is sutured to the surrounding host cartilage. The cells attach to the defect walls and produce the extracellular matrix in-situ. The major advantages of this method are the use of autologous tissue and the ability to expand the cell population. Difficulties with restoration of articular cartilage by this technique fall into three categories: cell adherence, phenotypic transformation, and ECM production.

Cell adherence. The success of transplantation of individual cells (without ECM) is critically dependent upon the cells attaching to the defect bed. Cartilage ECM has been shown to have anti-adhesive properties, which are believed to be conferred by small proteoglycans, dermatan sulfate, and heparan sulfate. Normal chondrocytes possess cell-surface receptors for type II collagen (M. P. Fernandez, et al., J. Biol. Chem. 263, 5921-5925 (1988)) and hyaluronan (H. J. Hauselmann, et al., Am J Physiol 271, C742-52 (1996)), but it is not clear to what extent ex-vivo manipulated cells possess receptors for these matrix molecules that are functional.

Phenotypic transformation. During the process of expanding the cell population in-vitro, chondrocytes usually undergo phenotypic transformation or dedifferentiation (K. Von Der Mark, Rheumatology 10, 272-315 (1986)). Morphologically, the cells resemble fibroblasts. Synthesis of type II collagen and aggrecan is diminished and synthesis of type I collagen, typical of fibrocartilage, is increased. Limited data exist to support the contention that the cells redifferentiate in-situ following transplantation. Reestablishment of the chondrocytic phenotype is critical to the success of the repair process, as tissue produced by cells which are phenotypically fibroblastic functions poorly as a replacement for articular cartilage.

ECM production. Prior to transplantation, the cultured chondrocytes are enzymatically denuded of ECM. The cells are injected into the defect bed as a suspension. The graft construct is incapable of bearing load and must be protected from weight bearing for several weeks to months which means long recovery time.

Each of the current methods of cartilage repair has substantial limitations. As a result, several laboratory approaches to production of cartilage tissue in-vitro have been proposed. These generally involve seeding of cultured cells (either chondrocytes or pluripotential stem cells) into a biological or synthetic scaffold.

U.S. Pat. No. 4,846,835 disclose a method for production of cartilage tissue in-vitro. Chondrocytes taken from a patient are multiplied in a mono-layer culture and, for further reproduction, are then introduced into a three-dimensional collagen matrix in the form of a gel or a sponge in which matrix they settle and become immobile. After about three weeks of cell reproduction, the defect cartilage location is filled with the material consisting of the collagen matrix and the cells. In order to hold the transplant in the defect location, a piece of periosteum is sutured over it. The cartilage regeneration in the region of this kind of transplant is considerably better than without the transplant.

U.S. Pat. No. 4,963,489 describes a similar method where a three-dimensional, artificial matrix is used as carrier material for the transplant. This matrix is used for the cell culture preceding the transplantation and is covered with a layer of connective tissue for better adhesion and better supply of the cells to be cultivated. After in-vitro cell reproduction on the three-dimensional matrix, the matrix is transplanted. The transplanted cells form the cartilage tissue in-vivo.

Biomaterials Vol. 17, No. 10, May 1996, Guilford suggests introducing vital cells into a tree-dimensional matrix for growing cartilage in-vitro and to then enclose the loaded matrix into a semi-permeable membrane. During the cartilage growth, this membrane is to prevent the culture medium to wash away compounds produced by the cells and being used for constructing the extracellular matrix. Transplantation of cell cultures enclosed in this kind of membranes is also known for preventing immune reactions.

US2014044682 discloses methods and compositions for treatment of an individual in need of cartilage repair. More particularly, fibroblasts or stem cells from an individual are harvested and cultured. The fibroblasts are then subjected to conditions that facilitate chondrocyte differentiation, such as low oxygen, mechanical stress, or a combination thereof. The chondrocytes are then provided to a mold which has the same shape as the damaged cartilage tissue.

WO2009111390 discloses methods of fabricating tissue engineered constructs comprising providing a cell sample comprising a plurality of chondrocytes, culturing the cell sample to produce a tissue-engineered cartilage construct, and treating the tissue-engineered cartilage construct, wherein treating the tissue-engineered cartilage construct comprises the use of a biochemical reagent, a mechanical force, hydrostatic pressure, or any combination thereof.

The major drawbacks of the above technologies, which are based on seeding of cultured cells into a biological or synthetic scaffold, are unknown biological effects of the scaffold material on the transplanted and native chondrocytes and other joint tissues and limited attachment of the engineered tissue construct to the defect bed. Since the transplant includes material that is not naturally present in the cartilage tissue there is also a question whether the transplant will facilitate formation of cartilage tissue having mechanical properties consistent with those of existing cartilage. Further, the use of support materials may be disadvantageous in that decomposition products thereof may affect other tissues, and when using non-autogenous support materials immune reactions or infections with animal or human pathogens may arise. Further, there is no disclosure of a cohesive cartilage construct being formed which has mechanical properties consistent with those of existing cartilage both in terms of compressive strength and lack of friction.

Thus, there is a need for a transplantable cohesive cartilage construct that does not suffer from the above drawbacks and which has mechanical properties consistent with those of existing cartilage both in terms of compressive strength and lack of friction.

### SUMMARY OF THE INVENTION

A first aspect of the present invention relates to a method for in-vitro production of a cohesive cartilage construct, the method comprising the following steps:
a) propagating chondrogenic cells derived from a subject to allow formation of one or more cartilage micro constructs;
b) putting a plurality of the cartilage micro constructs in motion to facilitate contact between the cartilage micro constructs and thereby allow formation of one or more fused cartilage micro constructs; and
c) subjecting one or more of the fused cartilage micro constructs to mechanical stimulation in a hypoxic environment to allow formation of a cohesive cartilage construct.

According to one embodiment, the chondrogenic cells are chondrocytes and in particular articular chondrocytes.

In one embodiment, the subject is a human or a non-human animal. Preferably the subject is a human.

According to one embodiment, the cells in step a) are propagated in a hypoxic environment. The hypoxic environment preferably being an environment with a partial pressure of molecular oxygen (O₂) less than 20%, such as less than 18%, less than 16%, less than 14%, less than 12%, less than 10%, less than 8%, less than 6%, less than 5%, less than 4%, less than 3% or less than 2.5%.

In one particularly preferred embodiment the hypoxic environment is an environment with a partial pressure of molecular oxygen (O₂) less than 10%.

According to one embodiment, the plurality of the cartilage micro constructs in step b) are put in motion in a hypoxic environment. The hypoxic environment preferably being an environment with a partial pressure of molecular oxygen (O₂) less than 20%, such as less than 18%, less than 16%, less than 14%, less than 12%, less than 10%, less than 8%, less than 6%, less than 5%, less than 4%, less than 3% or less than 2.5%.

In one particularly preferred embodiment the hypoxic environment is an environment with a partial pressure of molecular oxygen (O₂) less than 10%.

In one embodiment according to the present invention, the hypoxic environment is an environment with a partial pressure of molecular oxygen (O₂) less than 20%, such as less than 18%, less than 16%, less than 14%, less than 12%, less than 10%, less than 8%, less than 6%, less than 5%, less than 4%, less than 3% or less than 2.5%. In one particularly preferred embodiment the hypoxic environment is an environment with a partial pressure of molecular oxygen (O₂) less than 10%.

According to one embodiment, the chondrogenic cells in step a) are propagated by a technique, such as hanging drop cultivation, suitable to allow formation of one or more three-dimensional cell structures.

In one particularly preferred embodiment the one or more cartilage micro constructs referred to in step a) are three-dimensional cell structures, such as spheroids.

According to one embodiment, the chondrogenic cells in step a) and/or the plurality of the cartilage micro constructs in step b) and/or the one or more fused cartilage micro constructs in step c) are submerged in a cell culture media. In another embodiment the chondrogenic cells in step a) and/or the plurality of the cartilage micro constructs in step b) and/or the one or more fused cartilage micro constructs in step c) are contained in a cell culture media.

In a further embodiment according to the present invention, the chondrogenic cells in step a) are propagated in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In a further embodiment according to the present invention, the plurality of the cartilage micro constructs in step b) is contained in a cell culture media or submerged in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In a further embodiment according to the present invention, the fused cartilage micro constructs in step c) is contained in a cell culture media or submerged in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In a particularly preferred embodiment, the chondrogenic cells in step a) and/or the plurality of the cartilage micro constructs in step b) and/or the one or more fused cartilage micro constructs in step c) are submerged in a cell culture media or contained in a cell culture media; and the amount of dissolved molecular oxygen in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In one embodiment, the plurality of cartilage micro constructs in step b) are put in circular motion, such as uniform circular motion or smooth uniform circular motion, to facilitate contact between the cartilage micro constructs.

In another embodiment, the plurality of cartilage micro constructs in step b) are put in motion by subjecting the plurality of cartilage micro constructs in step b) to tilting about one axis to facilitate contact between the cartilage micro constructs. The tilting about one axis is achievable e.g. by using a shaker, such as a rocker shaker or a mini rocker shaker.

In another embodiment, the plurality of cartilage micro constructs in step b) are put in motion by subjecting the plurality of cartilage micro constructs in step b) to tilting about two or more axis, such as more than two independent horizontal axes, to facilitate contact between the cartilage micro constructs.

In one embodiment according to the present invention, step b) and step c) are combined into a one step process. This may be done e.g. by subjecting a plurality of the cartilage micro constructs obtained in step a) to mechanical stimulation in a hypoxic environment and put the plurality of the cartilage micro constructs in motion in a hypoxic environment to facilitate contact between the cartilage micro constructs and thereby allow formation of a cohesive cartilage construct.

In one embodiment according to the present invention, the mechanical stimulation is selected from the group consisting of compression, tension, oscillatory and/or vibrational stimulation, shear stress and any combination thereof.

Compression may be applied directly to the fused cartilage micro constructs; and/or the one or more fused cartilage micro constructs in step c) may be submerged in a cell culture media or contained in a cell culture media and the compression is applied to the surrounding cell culture media.

Tension may be applied biaxially and/or uniaxially resulting in a temporary structural deformation of the fused cartilage micro constructs.

Oscillatory and/or vibrational stimulation may applied directly to the fused cartilage micro constructs; and/or the one or more fused cartilage micro constructs in step c) are submerged or contained in a cell culture media and the oscillatory and/or vibrational stimulation is applied to the surrounding cell culture media.

In one particularly preferred embodiment, the mechanical stimulation is hydrodynamic stimulation.

In one embodiment according to the present invention, the one or more fused cartilage micro constructs in step c) are submerged in or contained in a cell culture media and the mechanical stimulation is hydrodynamic stimulation.

In one embodiment according to the present invention, the mechanical stimulation does not involve exposing the fused cartilage micro constructs and/or cohesive cartilage construct to a pressure ≥12 MPa.

In one embodiment according to the present invention, the mechanical stimulation does not involve exposing the fused cartilage micro constructs and/or cohesive cartilage construct to a pressure >10 MPa.

In one embodiment according to the present invention, the mechanical stimulation does not involve exposing the fused cartilage micro constructs and/or cohesive cartilage construct to a pressure ≥8 MPa.

In one embodiment according to the present invention, the mechanical stimulation does not involve exposing the fused cartilage micro constructs and/or cohesive cartilage construct to a pressure ≥7 MPa.

In one embodiment according to the present invention, the mechanical stimulation does not involve exposing the fused cartilage micro constructs and/or cohesive cartilage construct to a pressure ≥5 MPa.

In one particularly preferred embodiment, the hypoxic environment is an environment with a partial pressure of molecular oxygen (O₂) less than 10%; and the chondrogenic cells in step a) are propagated by a technique, such as hanging drop cultivation, suitable to allow formation of one or more three-dimensional cell structures.

In one particularly preferred embodiment,
the hypoxic environment is an environment with a partial pressure of molecular oxygen (O₂) less than 10%; and
the chondrogenic cells in step a) are propagated by a technique, such as hanging drop cultivation, suitable to allow formation of one or more three-dimensional cell structures; and
the one or more cartilage micro constructs referred to in step a) are three-dimensional cell structures, such as spheroids.

In one particularly preferred embodiment,
the cells in step a) are propagated in a hypoxic environment;
the hypoxic environment is an environment with a partial pressure of molecular oxygen (O₂) less than 10%; and
the chondrogenic cells in step a) are propagated by a technique, such as hanging drop cultivation, suitable to allow formation of one or more three-dimensional cell structures.

The mechanical stimulation may e.g. be a process involving a pressure which varies with time. In case the mechanical stimulation is a process involving a pressure which varies with time, the pressure which varies with time has a maximum pressure of less than 5 MPa, such as less than 4 MPa, less than 3 MPa, less than 2 MPa or less than 1.5 MPa.

In one embodiment, the fused cartilage constructs are subjected to a pressure of less than 5 MPa, such as less than 4 MPa, less than 3 MPa, less than 2 MPa or less than 1.5 MPa. Said pressure preferably being the mechanical stimulation.

In another embodiment, the mechanical stimulation involves a pressure which varies with time, provided that the maximum pressure is less than 12 MPa, such as ≤10 MPa.

The mechanical stimulation may e.g. be a process involving a pressure which varies with time. In case the mechanical stimulation is a process involving a pressure which varies with time, the pressure which varies with time has a maximum pressure of less than 12 MPa, such as ≤10 MPa, less than 8 MPa, less than 7 MPa or less than 5 MPa.

In one embodiment, the fused cartilage micro constructs are subjected to a pressure of less than 12 MPa, such ≤10 MPa, less than 8 MPa, less than 7 MPa or less than 5 MPa. Said pressure preferably being the mechanical stimulation.

In one embodiment, the mechanical stimulation involves a pressure which varies with time, provided that the maximum pressure is less than 10 MPa.

In one embodiment according to the present invention, the cohesive cartilage construct obtained in step c) contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

In one embodiment according to the present invention, the method does not involve seeding of chondrogenic cells into a biological or synthetic scaffold.

A second aspect of the present invention relates to a cohesive cartilage construct produced by the method according to the first aspect of the present invention.

In one embodiment according to the second aspect of the present invention, the cohesive tissue construct is substantially homogeneous cohesive cartilage construct.

In one embodiment according to the second aspect of the present invention, the cohesive cartilage construct contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

In one embodiment according to the present invention, the cohesive cartilage construct comprises extracellular matrix, substantially all of the extracellular matrix being produced by the cells of the cohesive cartilage construct.

In one embodiment according to the present invention, all cells of the cohesive cartilage construct are derived from the chondrogenic cells referred to in step a).

A third aspect of the present invention relates to the cohesive cartilage construct according to the second aspect of the present invention, for use in a surgical method for repairing damaged cartilage in a subject; the surgical method comprising the following step(s): replacing the damaged cartilage in the subject by removing the damaged cartilage and transplanting the cohesive cartilage construct.

In one embodiment according to the third aspect of the present invention, the subject from which the cells of the cohesive cartilage construct are derived is the subject into which the cohesive cartilage construct is transplanted.

In one embodiment according to the third aspect of the present invention, the damaged cartilage is damaged articular cartilage.

In one embodiment according to the third aspect of the present invention, subchondral bone at the site of the damaged cartilage is
penetrated to create a bleeding from blood vessels on top of the subchondral bone; and/or
scratched to create a minor bleeding from blood vessels on top of the subchondral bone;

after damaged cartilage has been removed but prior to transplanting the cohesive cartilage construct.

In one embodiment according to the third aspect of the present invention, the transplanted cohesive cartilage construct is sutured to surrounding cartilage and/or bone.

In one embodiment according to the third aspect of the present invention, the cause of the damaged cartilage is a degenerative disease, such as osteoarthritis.

An alternative aspect of the present invention relates to a method for in-vitro production of a cohesive tissue construct, the method comprising the following steps:
a) propagating cells derived from a subject to allow formation of a micro construct;
b) subjecting a plurality of the micro constructs to gentle movement to allow formation of a fused micro construct; and
c) subjecting the fused micro constructs to mechanical stimulation to allow formation of a cohesive tissue construct.

According to one embodiment according to the alternative aspect, the cells are chondrogenic cells, such as chondrocytes and in particular articular chondrocytes; the micro construct is cartilage micro construct, the fused micro construct is cartilage fused micro construct; and the cohesive tissue construct is cohesive cartilage construct.

In one embodiment according to the alternative aspect, the subject is a human or a non-human animal. Preferably the subject is a human.

In one embodiment according to the alternative aspect, the micro construct is a three-dimensional cell structure.

In one embodiment according to the alternative aspect, the cells in step a) are propagated by a technique suitable to allow formation of a three-dimensional cell structure, i.e. that the micro construct is a three-dimensional cell structure.

In one embodiment according to the alternative aspect, the cells in step a) are propagated in a hypoxic environment. In another embodiment according to the alternative aspect, the plurality of micro constructs in step b) are subjected to gentle movement in a hypoxic environment. In yet another embodiment according to the alternative aspect the fused micro constructs in step c) are subjected to mechanical stimulation in a hypoxic environment.

In one embodiment according to the alternative aspect the cells in step a) are propagated in a hypoxic environment; the plurality of micro constructs in step b) are subjected to gentle movement in a hypoxic environment; and/or the fused micro constructs in step c) are subjected to mechanical stimulation in a hypoxic environment.

In a preferred embodiment according to the alternative aspect, the cells in step a) are propagated in a hypoxic environment; the plurality of micro constructs in step b) are subjected to gentle movement in a hypoxic environment; and the fused micro constructs in step c) are subjected to mechanical stimulation in a hypoxic environment.

In one embodiment according to the alternative aspect, the hypoxic environment is an environment with a partial pressure of molecular oxygen (O₂) less than 20%, such as less than 10%, less than 5%, less than 4%, less than 3% or less than 2.5%.

In one embodiment according to the alternative aspect, the cells in step a), the plurality of micro constructs in step b) and/or the fused micro constructs in step c) are submerged/in a cell culture media. In another embodiment according to the alternative aspect, the cells in step a), the plurality of micro constructs in step b) and/or the fused micro constructs in step c) are contained in a cell culture media.

In a further embodiment according to the alternative aspect, the cells in step a) are propagated in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In a further embodiment according to the alternative aspect, the micro constructs in step b) are subjected to gentle movement in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In a further embodiment according to the alternative aspect, the fused micro constructs in step c) are subjected to mechanical stimulation in a cell culture media, wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

In one embodiment according to the alternative aspect, step b) and step c) are combined into a one step process. Said in other words, that the micro construct obtained in step a) is subjected to gentle movement and mechanical stimulation to allow formation of a cohesive tissue construct.

In another embodiment according to the alternative aspect, propagating the cells in step a) is by a technique suitable to allow formation of a three-dimensional cell structure, such as hanging drop cultivation.

In one embodiment according to the alternative aspect, mechanical stimulation is hydrodynamic stimulation.

The mechanical stimulation may e.g. be a process involving a pressure which varies with time. In case the mechanical stimulation is a process involving a pressure which varies with time, the pressure which varies with time has a maximum pressure of less than 5 MPa, such as less than 4 MPa, less than 3 MPa, less than 2 MPa or less than 1.5 MPa.

In one embodiment according to the alternative aspect, the fused micro constructs are subjected to a pressure of less than 5 MPa, such as less than 4 MPa, less than 3 MPa, less than 2 MPa or less than 1.5 MPa. Said pressure preferably being the mechanical stimulation.

In another embodiment according to the alternative aspect, the mechanical stimulation involves a pressure which varies with time, provided that the maximum pressure is less than 12 MPa, such as ≤10 Mpa.

In another embodiment according to the alternative aspect, mechanical stimulation is hydrodynamic stimulation.

The mechanical stimulation may e.g. be a process involving a pressure which varies with time. In case the mechanical stimulation is a process involving a pressure which varies with time, the pressure which varies with time has a maximum pressure of less than 12 MPa, such as ≤10 MPa, less than 8 MPa, less than 7 MPa or less than 5 MPa.

In one embodiment according to the alternative aspect, the fused micro constructs are subjected to a pressure of less than 12 MPa, such ≤10 MPa, less than 8 MPa, less than 7 MPa or less than 5 MPa. Said pressure preferably being the mechanical stimulation.

In one embodiment according to the alternative aspect, the mechanical stimulation involves a pressure which varies with time, provided that the maximum pressure is less than 10 MPa.

In one embodiment according to the alternative aspect,
the cells are chondrocytes; the micro construct is cartilage micro construct; the fused micro construct is cartilage fused micro construct; and cohesive tissue construct is cohesive cartilage construct;
the micro construct is a three-dimensional cell structure; and
   the cells in step a) are propagated in a hypoxic environment;
   the plurality of micro constructs in step b) are subjected to gentle movement in a hypoxic environment; and/or
   the fused micro constructs in step c) are subjected to mechanical stimulation in a hypoxic environment.

In one embodiment according to the alternative aspect, the cohesive tissue construct obtained in step c) contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

A second alternative aspect of the present invention relates to a cohesive tissue construct produced by the method according to the alternative aspect of the present invention.

In one embodiment according to the second alternative aspect of the present invention, the cohesive tissue construct is substantially homogeneous cohesive tissue construct.

In one embodiment according to the second alternative aspect of the present invention, the cohesive tissue construct contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

In one embodiment according to the second alternative aspect of the present invention, the cohesive tissue construct comprises extracellular matrix, substantially all of the extracellular matrix being produced by the cells of the cohesive tissue construct.

In one embodiment according to the second alternative aspect of the present invention, all cells of the cohesive tissue construct are derived from the cells referred to in step a).

A third alternative aspect of the present invention relates to the cohesive tissue construct according to the second alternative aspect of the present invention, for use in a surgical method for repairing damaged tissue in a subject; the surgical method comprising the following step(s): replacing the damaged tissue in the subject by removing the damaged tissue and transplanting the cohesive tissue construct.

In one embodiment according to the third alternative aspect of the present invention, the subject from which the cells of the cohesive tissue construct are derived is the subject into which the cohesive tissue construct is transplanted.

In one embodiment according to the third alternative aspect of the present invention, the damaged cartilage is damaged articular cartilage.

In one embodiment according to the third alternative aspect of the present invention, subchondral bone at the site of the damaged cartilage is
penetrated to create a bleeding from blood vessels on top of the subchondral bone; and/or
scratched to create a minor bleeding from blood vessels on top of the subchondral bone;
   after damaged cartilage has been removed but prior to transplanting the cohesive cartilage construct.

In one embodiment according to the third alternative aspect of the present invention, the transplanted cohesive cartilage construct is sutured to surrounding cartilage and/or bone.

In one embodiment according to the third alternative aspect of the present invention, the cause of the damaged cartilage is a degenerative disease, such as osteoarthritis.

### BRIEF DESCRIPTION OF DRAWINGS

The present invention is described in detail by reference to the following figures:

FIG. 1 shows a 3D diagram of articular cartilage (2.1) and subchondral bone (2.2) with interdigitations (2.3) interlocking with trabecular bone (2.4).

FIG. 2 illustrates a device for mechanically stimulating fused micro constructs to allow formation of a cohesive construct, the device comprising a piston/rod with a piston displacement of 6.44 cc (1), rod bushing (2), cylinder/bore (3), container with flexible floor and ceiling (4), nutrients (5), semipermeable chamber (6), spheroids (7), chamber (9), lid (11), vacuum port (12) and a pressure transducer (13). The working volume of the chamber (8) being 540 cc and the chamber being filled with e.g. distilled water (10).

FIG. 3 shows a diagram of various forms of mechanical stimulation: Black arrows represent direction of applied forces. Compression (A) can be applied directly to a cell-seeded construct or directly to the surrounding fluid as hydrostatic pressure. Tension (B) can be applied biaxially or uniaxially resulting in a temporary structural deformation of cells. Oscillatory or vibrational stimulation (C) can be applied to a cell-seeded construct or directly to the surrounding medium. Laminar shear stress (D) is applied through fluid flow, often to the interior of a cell-seeded lumen. The fluid velocity profile shown by the curve and lines in the middle demonstrates the distribution of fluid speed within a lumen experiencing pure laminar flow.

FIG. 4a shows a defect in the cartilage on the femoral side of the knee (left picture), and a loose fragment of cartilage after the accident (right picture)

FIG. 4b shows how the loose sheet of cartilage illustrated in FIG. 4a is fastened by gently inducing bleeding from bone for adhesion of the sheet and sutures fastening the fragment to normal surrounding cartilage (left picture). The picture to the right is a magnetic resonance imaging (MRI) picture showing healing of the sutured fragment to bone and surrounding cartilage.

FIG. 5a shows the result of 20 cartilage micro constructs being propagated in culture media in a hypoxic environment (3% O₂) for about 24 hours (no cell motion during propagation). The cartilage micro constructs remain mainly separated, some have sticked together; but there is no self-assembly into one fused cartilage micro construct.

FIG. 5b shows the result of 20 cartilage micro constructs being propagated in culture media in a hypoxic environment (3% (2) for about 24 hours. The cartilage micro-constructs were put in motion during propagation. The cartilage micro constructs have self-assembled into one fused micro construct.

FIG. 6 shows the result of 47 cartilage micro constructs being propagated in culture media in a hypoxic environment (3% O₂). The cartilage micro-constructs were put in motion during propagation. The cartilage micro constructs have self-assembled into a fused micro construct and it is complete fusion between micro constructs. FIG. 6 has higher magnification than the pictures in FIGS. 5a and 5b.

FIG. 7 shows the result of 47 cartilage micro constructs being propagated in culture media in a hypoxic environment (3% O₂) for about 48 hours, wherein the cartilage micro constructs have been put in motion for 3 hours per day and thereafter subjected to mechanical stimulation 1 hour per day for another 48 hours. As demonstrated in FIG. 7, the cartilage micro constructs have self-assembled into a small cohesive cartilage construct (indicated by the black arrow) measuring about 6 mm in length and 2 mm in breadth.

FIG. 8 shows histological data based on the small cohesive cartilage construct depicted in FIG. 7. As can be seen from the histological data, the chondrocytes have created cohesive cell sheets indicating a transition from cell culture to a bona fide tissue. Some of the chondrocytes are already lying in the lacuna-like spaces (thin arrows), and there is focally seen forming of basophilic acid glycosaminoglycan-rich extracellular matrix (thick arrows) imparting the newly formed tissue resemblance to an immature cartilage.

### DETAILED DESCRIPTION OF THE INVENTION

Unless specifically defined herein, all technical and scientific terms used have the same meaning as commonly understood by a skilled artisan in the fields of genetics, biochemistry, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numerical limit or range is stated herein, the endpoints are included. Also, all values and sub ranges within a numerical limit or range are specifically included as if explicitly written out.

Martinez et al (Cell transplantation, vol. 17, pages 987-996, 2008) describes a culturing technique, which is not based on the use of supporting structures, for production of well-formed and solid cartilage micro constructs ranging from 200 to 600 μm in size. The micro constructs are derived from chondrocyte suspensions and claimed to share morphological and phenotypic similarities with native hyaline cartilage.

However, to the best of our knowledge there is no disclosure in the prior art of a culturing technique which facilitate fusion of the cartilage micro constructs of the prior art and thereby allow formation of a cohesive cartilage construct.

A first aspect and an alternative aspect of the present invention relates to a method for in-vitro production of a cohesive tissue construct, such as a cohesive cartilage construct. By being able to produce the cohesive tissue construct, such as a cohesive cartilage construct, patients suffering from tissue defect, such as cartilage defects, may be more efficiently treated and the recovery time is expected to be significantly reduced.

The cohesive cartilage construct is typically derived from cells that have been isolated from a human or a non-human animal. In principal, the cells may be any type of cell, but it is preferred that the cells are chondrogenic cells, such as chondrocytes and in particular articular chondrocytes. The chondrocytes may e.g. be obtained from a biopsy collected from an area of the knee which preferably is exposed to low stress. The person skilled in the art will be aware of a number of different techniques that successfully may be used to isolate such cells. One example of such a method is provided in Martinez et al (Cell transplantation, vol. 17, pages 987-996, 2008) on page 988, left column, last paragraph to page 988, right column, first paragraph. Reference is also made to example 1 of the present application disclosing a method for isolating and propagating human articular chondrocytes.

### Step a)

Once the cells have been isolated, the cells are propagated for a period of time sufficient to allow formation of micro construct(s), such as cartilage micro construct(s). The person skilled in the art will know how to propagate the cells in order to obtain (cartilage) micro construct(s). One example of such a method is provided in Martinez et al (Cell transplantation, vol. 17, pages 987-996, 2008) on page 989, left column, second section. Reference is also made to example 2 of the present application disclosing hanging-drop cultivation of human articular chondrocytes.

In one embodiment according to the present invention, the (cartilage) micro construct(s) is three-dimensional cell structure(s), such as spheroid(s), and the cells are propagated to allow formation of three-dimensional cell structure(s). The term “three-dimensional cell structure(s)” as used herein refers to cells which have been allowed to grow in all three dimensions, similar to how they would in vivo. Hanging drop cultivation (Cell Transplantation 2008; 17:987-996) being one example of a technique suitable for three-dimensional cultivation of cells.

Thus, in a preferred embodiment according to the present invention the cells are propagated by hanging-drop cultivation to allow formation of (cartilage) micro construct(s), each (cartilage) micro construct being a three-dimensional cell structure.

The hanging drop cultivation is a technique typically utilized in embryology and other fields to allow growth that would otherwise be restricted by the flat plane of culture dishes and also to minimize the surface area to volume ratio, slowing evaporation. The classic hanging drop culture is a small drop of liquid, such as plasma or some other media allowing tissue growth, suspended from an inverted watch glass. The hanging drop is then suspended by gravity and surface tension, rather than spreading across a plate. This allows tissues or other cell types to be examined without being squashed against a dish. This is useful when e.g. the three-dimensional structure of a tissue is desired. A detailed disclosure of hanging-drop cultivation of human articular chondrocytes is provided in example 2 of the present application.

In one embodiment according to the present invention, the cells are propagated by hanging-drop cultivation, the hanging-drop cultivation comprising the following step(s):
a drop of a suspension of cells, such as chondrogenic cells, is dispensed onto a surface, the cells of the cell suspension being the cells isolated from a human or non-human animal;
the surface is inverted; and
the cells are propagated to allow formation of (cartilage) micro construct(s), the (cartilage) micro construct(s) typically having a three-dimensional cell structure.

In one embodiment, the (cartilage) micro construct is spherical in shape. The size of each (cartilage) micro construct is typically in the range 200 to 1000 μm, such as in the range 200 to 900 μm, in the range 200 to 800 μm, in the range 200 to 700 μm, in the range 200 to 600 μm, in the range 200 to 500 μm or in the range 200 to 400 μm. Reference is made to Martinez et al (Cell transplantation, vol. 17, pages 987-996, 2008) for calculating the size of the micro construct.

If the number of cells per drop is too high, the spontaneous cell assembling may be hampered and the resulting structures may become less solid and little consistent. By using smaller number of cells it will be easier to feed the cells and also easier for the cells to get rid of waste products.

Thus, in one embodiment according to the present invention the number of cells in each drop is in the range 1000 cells/drop to 30 000 cells/drop, such as in the range 2000 cells/drop to 30 000 cells/drop, in the range 2000 cells/drop to 20 000, in the range 2000 cells/drop to 10 000, in the range 2000 cells/drop to 8000, in the range 2000 cells/drop to 5000, or 2000 cells/drop to 5000.

In another embodiment according to the present invention the average number of cells in each drop is in the range 1000 cells/drop to 30 000 cells/drop, such as in the range 2000 cells/drop to 30 000 cells/drop, in the range 2000 cells/drop to 20 000, in the range 2000 cells/drop to 10 000, in the range 2000 cells/drop to 8000, in the range 2000 cells/drop to 5000, or 2000 cells/drop to 5000.

The cells are typically propagated for an amount of time effective for allowing formation of (cartilage) micro constructs(s). The cells typically accumulate at the bottom of the drop after 24 hours. By day three the cells are typically more packed, and at day seven the cells have typically formed homogenous, well-rounded and solid aggregates. After 7 days there are typically nonsignificant changes with respect to aggregate size and shape, clearly indicating that a plateau in the growth has been reached. Viability of the cells are typically high up to day 21 of culture as measured by trypan blue exclusion assay. Said aggregates typically have the shape of spheres with a size in the range 200-600 μm. These aggregates are herein referred to as (cartilage) micro constructs.

It is to be understood that the amount of time effective for allowing formation of (cartilage) micro construct(s) may vary between cell types and may also vary depending on the culturing conditions. Thus, in one embodiment according to the present invention, the amount of time effective for allowing formation of (cartilage) micro construct(s) is in the range 1-21 days, preferably in the range 1-15 days, more preferably in the range 1-7 days such as in the range 3-7 days.

The oxygen requirements for optimal in-vitro propagation of cartilage cells should in theory get close to the hypoxic conditions encountered in native tissue. Thus, in one embodiment according to the present invention, the cells, such as chondrogenic cells, in step a) are propagated in a hypoxic environment.

Hypoxia refers to low oxygen conditions. About 20.9% of the gas in the atmosphere at 1 atm is molecular oxygen (i.e. the partial pressure of oxygen in the atmosphere is 20.9% of the total barometric pressure). If a sample is subjected to a hypoxic environment, the partial pressure of molecular oxygen in the surrounding air is typically less than 20.9%.

When the cells in step a) are subjected to hypoxic environment, the cells are typically cultivated in a culture media that is exposed to air where the partial pressure of molecular oxygen (O₂) is less than 20.9%. One way of obtaining such conditions is to incubate the cells in an incubator where the percentage of molecular oxygen inside the incubator is decreased as compared to the percentage of molecular oxygen outside the incubator at 1 atm.

In one embodiment, the hypoxic environment is an environment with a partial pressure of molecular oxygen less than 20%, such as less than 10%, less than 5%, less than 4%, less than 3% or less than 2.5%. In a preferred embodiment, the hypoxic environment is an environment with a partial pressure of molecular oxygen in the range 1 to 10%, such as in the range 1 to 8%, 1 to 6%, 1 to 4% or 2 to 4%. In a particularly preferred embodiment, the hypoxic environment is an environment with a partial pressure of molecular oxygen in the range 2 to 5%, such as in the range 2 to 4%.

By propagating cells in a hypoxic environment, the amount of molecular oxygen that is dissolved in the culturing medium will also decrease over time. Dissolved oxygen is typically expressed as a percentage of the oxygen that would dissolve in the water at the prevailing temperature and salinity (both of which affect the solubility of oxygen in water).

In a stable body of an aqueous solution with no stratification, dissolved oxygen will remain at 100% air saturation. 100% air saturation means that the water is holding as many dissolved gas molecules as it can in equilibrium. At equilibrium, the percentage of each gas in the water would be equivalent to the percentage of that gas in the atmosphere—i.e. its partial pressure. The water will slowly absorb oxygen and other gasses from the atmosphere until it reaches equilibrium at complete saturation.

Two bodies of water that are both 100% air-saturated do not necessarily have the same concentration of dissolved oxygen. The actual amount of dissolved oxygen (in mg/L) will vary depending on temperature, pressure and salinity.

First, the solubility of oxygen typically decreases as temperature increases. This means that warmer surface water requires less dissolved oxygen to reach 100% air saturation than does deeper, cooler water. Second, dissolved oxygen decreases exponentially as salt levels increase. That is why, at the same pressure and temperature, saltwater holds about 20% less dissolved oxygen than freshwater. Third, dissolved oxygen will increase as pressure increases. This is true of both atmospheric and hydrostatic pressures. Water at lower altitudes can hold more dissolved oxygen than water at higher altitudes. This relationship also explains the potential for “supersaturation” of waters below the thermocline—at greater hydrostatic pressures, water can hold more dissolved oxygen without it escaping.

In order to illustrate the solubility of oxygen under different conditions reference is made to table 1 below listing the maximum amount of dissolved molecular oxygen, i.e. 100% saturation, at different temperatures and salinities.

TABLE 1

| Temp ° C. | freshwater | saltwater |
|---|---|---|
| 0 | 14.6 | 11.7 |
| 5 | 12.8 | 10.4 |
| 10 | 11.3 | 9.3 |
| 15 | 10.1 | 8.5 |
| 20 | 9.1 | 7.8 |
| 25 | 8.2 | 7.1 |
| 30 | 7.5 | 6.5 |

### Maximum Level of Molecular Oxygen in Mg/L in Sterile Water at a Particular Temperature.

A freshwater solution left to stand in air at 1 atm at a temperature of 25° C. will have 8.2 mg/L dissolved molecular oxygen at full saturation, i.e. at 100% air saturation. At 50% air saturation under the same conditions, the freshwater solution would have 4.1 mg/L dissolved molecular oxygen.

The cells in step a), preferably the chondrogenic cells, are typically submerged or contained in a cell culture media during cultivation. The term “hypoxic environment” as used herein refers to the environment surrounding the cell culture media, i.e. it does not refer to the cell culture media per se but refers to the air which is surrounding the cell culture media. Thus, in a hypoxic environment it will take some time before the hypoxic environment will affect the amount of molecular oxygen that is dissolved in the cell culture media, i.e. that it will take some time to establish a new equilibrium.

Thus, in order to reduce the time to establish a new equilibrium, the culture medium may be subjected to treatment to reduce the amount of dissolved molecular oxygen before use. Said in other words, that the cells (preferably chondrogenic cells) in step a) are propagated in a culture media having an amount of dissolved molecular oxygen less than 100% air saturation. If you combine this with propagation in a hypoxic environment, the cells will experience a quick and prolonged drop in the amount of molecular oxygen dissolved in said culture media as compared to propagation in a hypoxic environment with culture media having 100% air saturation.

Thus, in one embodiment according to the present invention the cells, preferably chondrogenic cells, in step a) is propagated in a culture media wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation. In another embodiment, the amount of dissolved molecular oxygen in said cell culture media is in the range 1-30% air saturation, such as in the range 1-20%, in the range 1-10%, in the range 1-5% or in the range 2-3%.

Percentage air saturation represents a value that is directly linked to the characteristics of the air. In order to transform this value into an absolute value it is necessary to establish a reference point.

The term “culture media at 100% air saturation” as used herein refers to a culture media with an amount of dissolved molecular oxygen which corresponds to a culture media which has been left to stand in air at 1 atm at room temperature (about 20° C.) for sufficient time to establish an equilibrium, i.e. that the molecular oxygen is dissolved at full saturation. Such a culture media is typically prepared by providing culture media and let it stand on a lab bench for sufficient time to establish an equilibrium. A culture media where the amount of dissolved O₂ is at 80% air saturation is a culture media that has 20% less dissolved molecular oxygen as compared to a culture media at 100% air saturation at 1 atm and room temperature.

It is an object of the invention to produce a transplantable cohesive cartilage construct which has mechanical properties consistent with those of existing cartilage both in terms of compressive strength and lack of friction. Nearly 95 percent of articular cartilage is extracellular matrix (ECM) that is produced and maintained by the chondrocytes dispersed throughout it. The ECM consists mainly of proteoglycan and collagens and the main proteoglycan in cartilage is aggrecan. Aggrecan, as its name suggests, forms large aggregates with hyaluronan. These aggregates are negatively charged and hold water in the tissue. The collagen, mostly collagen type II, constrains the proteoglycans. The ECM responds to tensile and compressive forces that are experienced by the cartilage thereby maintaining the mechanical integrity of the tissue.

Thus, in one embodiment according to the present invention the cells, preferably chondrogenic cells, in step a) are propagated to allow formation of micro construct(s), such as cartilage micro construct(s), which contain at least 40% by volume of extracellular matrix, such as at least 60% by volume of extracellular matrix, at least 80% by volume of extracellular matrix, at least 90% by volume of extracellular matrix such as about 95% by volume of extracellular matrix.

### Step b)

The second step of this method involves:
subjecting a plurality of the micro constructs, such as cartilage micro constructs, obtained in step a) to gentle movement to allow formation of a fused micro construct, such as fused cartilage micro construct; or
putting a plurality of the cartilage micro constructs in motion to facilitate contact between the cartilage micro constructs and thereby allow formation of one or more fused cartilage micro constructs.

According to one embodiment, the plurality of the cartilage micro constructs in step b) are put in motion in a hypoxic environment. The hypoxic environment preferably being an environment with a partial pressure of molecular oxygen (O₂) less than 20%, such as less than 18%, less than 16%, less than 14%, less than 12%, less than 10%, less than 8%, less than 6%, less than 5%, less than 4%, less than 3% or less than 2.5%.

In one particularly preferred embodiment the hypoxic environment is an environment with a partial pressure of molecular oxygen (O₂) less than 10%.

The term “fused (cartilage) micro construct” as used herein refers to at least two (cartilage) micro constructs which have “sticked” together, self-assembled into a fused (cartilage) micro construct and wherein there is fusion between the (cartilage) micro constructs. An example of a fused (cartilage) micro construct which is the result 47 (cartilage) micro constructs that have “sticked together”, self-assembled into a fused (cartilage) micro construct and wherein there is fusion between the (cartilage) micro constructs is provided in FIG. 6.

It has previously been disclosed how to prepare well-formed and solid cartilage micro constructs ranging from 200 to 600 μm in size (Cell transplantation, vol. 17, pages 987-996, 2008). The cartilage micro constructs are derived from chondrocyte suspensions and share morphological and phenotypic similarities with native hyaline cartilage. However, to the best of our knowledge it has proven difficult to facilitate fusion of these cartilage micro constructs into fused cartilage micro constructs. The formation of a fused (cartilage) micro construct is believed to be important for production of a cohesive cartilage construct which has mechanical properties consistent with those of existing cartilage both in terms of compressive strength and lack of friction.

Without being bound by theory, it was assumed that the (cartilage) micro constructs formed in step a) have the ability to stick to each other under the right conditions. Further, it was hypothesized that such close contact would result in a fusion between (cartilage) micro constructs thereby forming a fused (cartilage) micro construct.

A prerequisite for the (cartilage) micro constructs to “stick together” is that the (cartilage) micro constructs are brought into contact with each other. The more often the (cartilage) micro constructs are brought into contact with each other, the more likely they are to stick together and finally fuse into one fused (cartilage) micro construct.

Based on this theory, it was decided to
subject a plurality of the micro constructs to gentle movement; or
put a plurality of the cartilage micro constructs in motion to facilitate contact between the cartilage micro constructs;

by placing the container harboring the (cartilage) micro constructs onto a mini rocker shaker, cf. example 4. The mini rocker shaker was started with a 10 degrees slope and it took about 15 seconds to reach 0 degrees, another 15 seconds to reach-10 degrees slope, another 15 seconds to reach 0 degrees and another 15 seconds to return to start at a 10 degrees slope; thus a total of 30 seconds from 10 degrees slope to −10 degrees slope and a total of 60 seconds from start of movement until a full turn had been obtained.

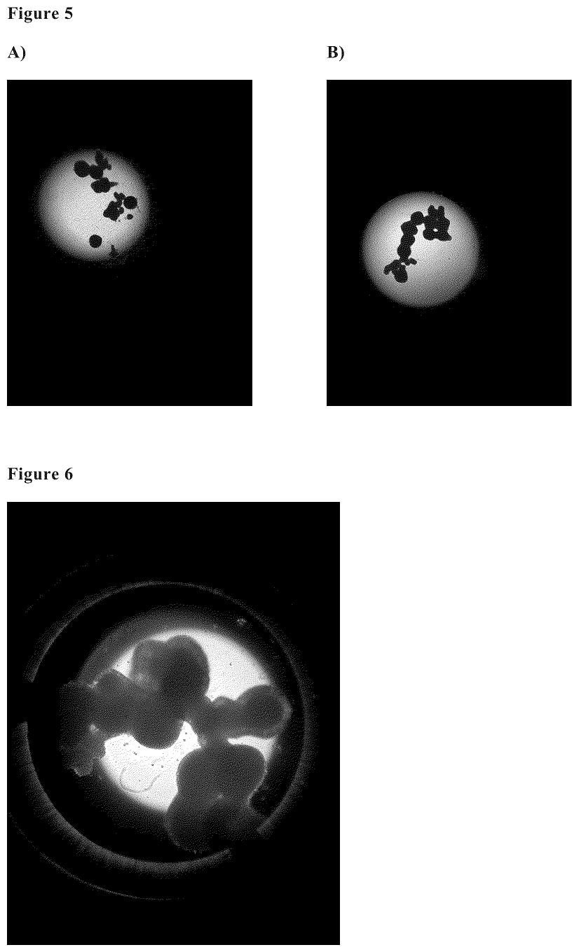

Surprisingly, the inventor of the present invention was able to show that this kind of movement significantly increased the chances of the formation of fused (cartilage) micro construct(s). FIG. 5a clearly demonstrates that if the (cartilage) micro constructs are not subjected to movement or put in motion, the (cartilage) micro constructs remain mainly separated, some have sticked together; but there is no self-assembly into one fused (cartilage) micro construct as can be seen in FIG. 5b. If the (cartilage) micro constructs are subjected to gentle movement or put in motion, the (cartilage) micro constructs will self-assemble into one fused (cartilage) micro construct as shown in FIG. 5b.

It is believed that subjecting a plurality of the micro constructs to gentle movement or putting a plurality of the cartilage micro constructs in motion is important in order for the (cartilage) micro constructs to stick together. If the (cartilage) micro constructs are subjected to powerful movement, it was initially believed that the (cartilage) micro constructs will not have sufficient time to stick together and that they rather will hit and leave like pool balls. Thus, the term “gentle movement” as used herein refers to movement which provides the (cartilage) micro constructs with sufficient time to stick together and at the same time do not provide too powerful movement to cause hit and leave functionality as referred to above.

Even though powerful movement may not be the preferred option, it is now believed that the strength of the movement is of less importance. The most important feature seems to be that the plurality of (cartilage) micro constructs are put in motion to facilitate contact between the (cartilage) micro constructs. Thus, the number of times a (cartilage) micro construct makes contact with other micro constructs seems to be more important than the speed of the micro constructs at the time of contact.

If e.g. a mini rocker shaker is used (provides movement in the form of tilting about one axis) to put the plurality of (cartilage) micro constructs in motion, the person skilled in the art will understand that if the speed of the mini rocker shaker is too high, the (cartilage) micro constructs may not have sufficient time to make contact with other (cartilage) micro constructs.

In example 4 a mini rocker shaker was used to provide the gentle movement. This mini rocker shaker provided only movement in the form of tilting about one axis. Based on the hypothesis that was raised above and the achieved results, it seems reasonable to assume that the result may have been even better if the mini rocker shaker would have been able to provide movement in the form of tilting about more than two independent horizontal axes. One example of a device which would provide movement in the form of tilting about more than two independent horizontal axes is the so-called orbital shaker.

In one embodiment according to the present invention, the cells referred to in step a) are chondrogenic cells, such as chondrocytes; the micro construct(s) is cartilage micro construct(s) and the fused micro construct(s) referred to in step b) are fused cartilage micro construct(s).

The oxygen requirements for optimal in-vitro fusion of (cartilage) micro constructs should in theory get close to the hypoxic conditions encountered in native tissue. Thus, in one embodiment according to the present invention, the (cartilage) micro construct(s) in step b) are
subjected to gentle movement in a hypoxic environment; or
put in motion to facilitate contact between the (cartilage) micro constructs in a hypoxic environment.

Hypoxia refers to low oxygen conditions. About 20.9% of the gas in the atmosphere at 1 atm is molecular oxygen (the partial pressure of oxygen in the atmosphere is 20.9% of the total barometric pressure). If a sample is subjected to a hypoxic environment, the partial pressure of oxygen in the surrounding air is typically less than 20.9%.

When the (cartilage) micro constructs in step b) are subjected to hypoxic environment, the micro constructs are typically cultivated in a culture media that is exposed to air where the partial pressure of molecular oxygen (O₂) is less than 20.9%. One way of obtaining such conditions is to incubate the (cartilage) micro constructs in an incubator where the percentage of molecular oxygen inside the incubator is decreased as compared to the percentage of molecular oxygen outside the incubator at 1 atm.

In one embodiment, the hypoxic environment is an environment with a partial pressure of molecular oxygen less than 20%, such as less than 10%, less than 5%, less than 4%, less than 3% or less than 2.5%. In a preferred embodiment, the hypoxic environment is an environment with a partial pressure of molecular oxygen in the range 1 to 10%, such as in the range 1 to 8%, 1 to 6%, 1 to 4% or 2 to 4%. In a particularly preferred embodiment, the hypoxic environment is an environment with a partial pressure of molecular oxygen in the range 2 to 5%, such as in the range 2 to 4%.

The (cartilage) micro constructs in step b) are typically submerged or contained in a culture medium. By keeping the (cartilage) micro constructs in a hypoxic environment, the amount of molecular oxygen that is dissolved in the culturing medium will also decrease over time.

The term “hypoxic environment” as used herein refers to the environment surrounding the cell culture media, i.e. it does not refer to the cell culture media per se but refers to the air which is surrounding the cell culture media. Thus, in a hypoxic environment it will take some time before the hypoxic environment will affect the amount of molecular oxygen that is dissolved in the cell culture media, i.e. that it will take some time to establish a new equilibrium.

Thus, in order to reduce the time to establish a new equilibrium, the culture medium may be subjected to treatment to reduce the amount of dissolved molecular oxygen before use. Said in other words, that the (cartilage) micro constructs in step b) are subjected to gentle movement in a culture media having an amount of dissolved molecular oxygen less than 100% air saturation. If you combine this with keeping the (cartilage) micro constructs in a hypoxic environment, the micro constructs will experience a quick and prolonged drop in the amount of molecular oxygen dissolved in said culture media.

Thus, in one embodiment according to the present invention the (cartilage) micro constructs in step b) are subjected to gentle movement in a culture media wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation. In another embodiment, the amount of dissolved molecular oxygen in said cell culture media is in the range 1-30% air saturation, such as in the range 1-20%, in the range 1-10%, in the range 1-5% or in the range 2-3%.

Percentage air saturation represents a value that is directly linked to the characteristics of the surrounding air. In order to transform this value into an absolute value it is necessary to establish a reference point.

The term “culture media at 100% air saturation” as used herein refers to a culture media with an amount of dissolved molecular oxygen which corresponds to a culture media which has been left to stand in air at 1 atm at room temperature (about 20° C.) for sufficient time to establish an equilibrium, i.e. that the molecular oxygen is dissolved at full saturation. Such a culture media is typically prepared by providing culture media and let it stand on a lab bench for enough time to establish an equilibrium. A culture media where the amount of dissolved O₂ is at 80% air saturation is a culture media that has 20% less dissolved molecular oxygen as compared to a culture media at 100% air saturation at 1 atm and room temperature.

In one embodiment according to the present invention, the plurality of the micro constructs represents at least 3 micro constructs, such as at least 5 micro constructs, at least 10 micro constructs, at least 20 micro constructs, at least 30 micro constructs, at least 40 micro constructs, at least 50 micro constructs, at least 60 micro constructs, at least 80 micro constructs, at least 100 micro constructs, at least 120 micro constructs, at least 140 micro constructs, at least 160 micro constructs, at least 180 micro constructs or at least 200 micro constructs.

In another embodiment according to the present invention the plurality of the micro constructs is in the range 10-200 micro constructs, such as in the range 10-100 micro constructs, in the range 10-80 micro constructs, in the range 10-60 micro constructs, in the range 10-40 micro constructs in the range 10-20 micro constructs or in the range 20-100 micro constructs.

It is an object of the invention to produce a transplantable cohesive cartilage construct which has mechanical properties consistent with those of existing cartilage both in terms of compressive strength and lack of friction.

Thus, in one embodiment according to the present invention the (cartilage) micro constructs obtained in step b) are subjected to gentle movement to allow formation of fused (cartilage) micro construct(s) which contain at least 40% by volume of extracellular matrix, such as at least 60% by volume of extracellular matrix, at least 80% by volume of extracellular matrix, at least 90% by volume of extracellular matrix such as about 95% by volume of extracellular matrix.

### Step c)

The third step of the method according to the present invention involves subjecting the fused (cartilage) micro construct(s) obtained in step b) to mechanical stimulation, preferably in a hypoxic environment, to allow formation of a cohesive tissue construct, such as a cohesive cartilage construct.

In one embodiment according to the present invention, the cells of step a) are chondrocytes and the micro constructs obtained in step a) are cartilage micro constructs, the fused micro constructs obtained in step b) are fused cartilage micro constructs; and the cohesive tissue construct obtained in step c) is cohesive cartilage construct.

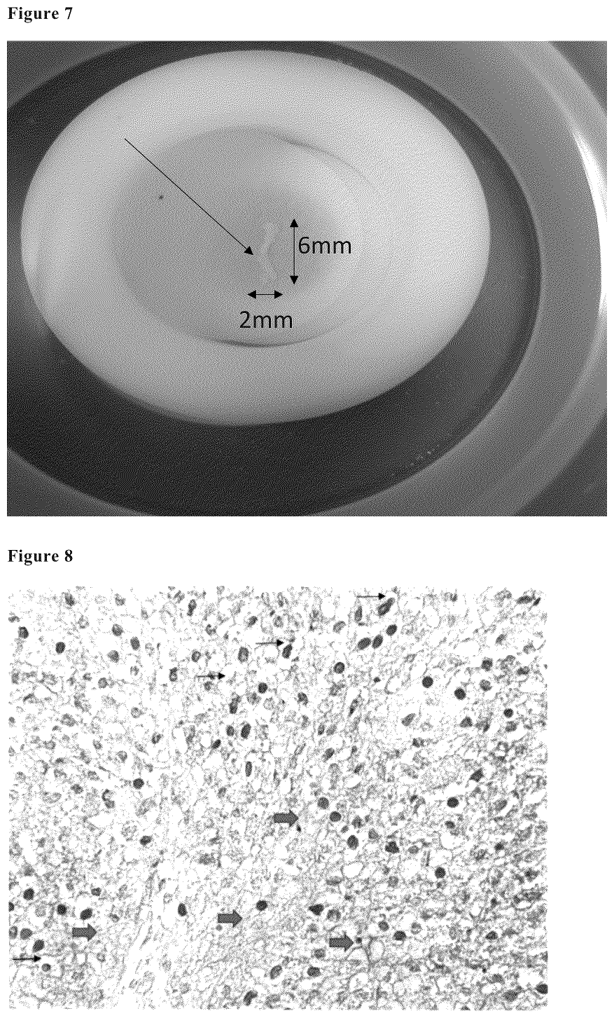

The term “cohesive cartilage construct” as used herein refers to a transplantable cohesive cartilage construct which has mechanical properties consistent with those of existing cartilage both in terms of compressive strength and/or lack of friction. An example of a cohesive cartilage construct is provided in FIG. 7.

Based on the idea that the physiological forces that chondrocytes are subjected to in-vivo may be important for the development of tissue engineered cartilage, the inventor of the present invention started to investigate the effect of mechanical stimulation applied during in-vitro tissue formation.

A bioreactor (FIG. 2) capable of transmitting hydrodynamic forces to the fused (cartilage) micro construct(s) in vitro was developed, and the effect of mechanical stimulation on the development and the properties of the fused (cartilage) micro construct(s) were determined (example 3). Surprisingly it was observed that the fused (cartilage) micro constructs developed into a cohesive tissue construct, such as a cohesive cartilage construct, that was larger in size compared to prior art constructs and shared morphological and phenotypic similarities with native cartilage (see FIG. 8).

Thus, it one embodiment according to the present invention mechanical stimulation is hydrodynamic stimulation. The term “hydrodynamic stimulation” as disclosed herein refers to a situation where hydrodynamic forces are applied to the fused (cartilage) micro construct(s). An example of a procedure in which the fused (cartilage) micro construct(s) is subjected to hydrodynamic stimulation is described in example 3 of the present application.

In one embodiment according to the present invention, the fused (cartilage) micro construct is placed inside a first container (6) filled with culture media. The floor and/or the ceiling of the first container (6) are preferably semipermeable membranes.

The term “semipermeable membrane” refers to biological or synthetic, polymeric membrane that will allow certain molecules or ions to pass through it by diffusion by more specialized processes of facilitated diffusion, passive transport or active transport. In one embodiment, the semipermeable membrane is a biological or synthetic membrane, preferably synthetic, which allows nutrients and waste products to pass through it. The rate of passage will typically depend on the pressure, concentration, and temperature of the molecules or solutes on either side, as well as the permeability of the membrane to each solute. The purpose of the semipermeable membrane being to allow transport of nutrients and waste products and at the same time to transmit hydrodynamic forces that is applied to the membrane.

The first container is preferably placed inside a second container (4). The second container is filled with a liquid, said liquid preferably being a culture media; and most preferably the culture media of the first container and the culture media of the second container are the same. At least one of the faces, such as two of the faces, of the second container being an impermeable membrane.

The term “impermeable membrane” as used herein refers to a biological or synthetic membrane which does not allow molecules or ions to pass through it. The purpose of the impermeable membrane is to transmit hydrodynamic forces that is applied to the membrane.

The second container is preferably placed inside a bioreactor-container (9). The bioreactor-container is filled with a liquid, said liquid preferably being distilled water. The bioreactor-container preferably having the shape of a circular cylinder. All faces of the bioreactor-chamber are preferably of a non-flexible material. The liquid inside the bioreactor-chamber is preferably in fluid communication with means suitable for inducing a change in pressure inside the bioreactor-chamber, such as a piston (1).

During mechanical stimulation, an energy in the range 0.5 Mpa to 50 MPa, such as in the range 1 Mpa to 10 MPa, in the range 1 Mpa to 5 MPa or in the range 1 Mpa to 4 MPa is applied, e.g. to the piston, which results in an increased pressure inside the bioreactor-container. Optionally, an energy in the range 0.5 Mpa to 12 MPa, such as in the range 1 Mpa to 12 MPa, in the range 1 Mpa to 10 MPa or in the range 1 Mpa to 5 MPa is applied, e.g. to the piston during mechanical stimulation.

The change in pressure inside the bioreactor-container, i.e. the hydrodynamic force, will be transmitted over the impermeable membrane of the second container. Further, the change in pressure inside the second container, i.e. the hydrodynamic force, will be transmitted over the semipermeable membrane of the first container where the cartilage micro constructs are placed. After a predetermined period of time, the energy applied to the means suitable for inducing a change in pressure inside the bioreactor-chamber is reduced resulting in a decreased pressure inside the bioreactor-chamber.

In one embodiment according to the present invention, the change in energy applied to the means suitable for inducing a change in pressure inside the bioreactor-chamber is an oscillatory change in pressure. Thus, in one embodiment according to the present invention, mechanical stimulation is oscillatory hydrodynamic stimulation.

The term “oscillatory hydrodynamic stimulation” refers to the repetitive variation in time of the hydrodynamic force, e.g. energy applied to the means suitable for inducing a change in pressure inside the bioreactor-chamber, about a central value or between two different states.

In another embodiment according to the present invention, the change in energy applied to the means suitable for inducing a change in pressure inside the bioreactor-chamber is a pulsating change in pressure. Thus in one embodiment according to the present invention, mechanical stimulation is pulsating hydrodynamic stimulation.

The term “pulsating hydrodynamic stimulation” refers to the periodic variations in time of the hydrodynamic force. The pulse may be irregular, i.e. non-repetitive with time, or may be regular, i.e. repetitive with time.

In one embodiment according to the present invention, the mechanical stimulation is oscillatory or pulsating mechanical stimulation. Pulsating mechanical stimulation may be irregular pulsating mechanical stimulation or regular pulsating mechanical stimulation.

In another embodiment according to the present invention, the mechanical stimulation is a change in pressure. The change in pressure may be an oscillatory change in pressure or a pulsating change in pressure. In case of a pulsating change in pressure, the pulse may be irregular or regular.

In one embodiment according to the present invention, the mechanical stimulation is a compressive load, such as a uniaxial compressive load, with x MPa, y Hz for z hour, wherein
x is in the range 0.5 to 50, such as in the range 1 to 40, such as in the range 1 to 30, such as in the range 1 to 20 or in the range 1 to 10.
y is in the range 0.1 to 2, such as in the range 0.5 to 1.5 or about 1; and
z is in the range 50 to 400, 50 to 300, 60 to 300, 70 to 250, 20 to 60 or 72 to 240.

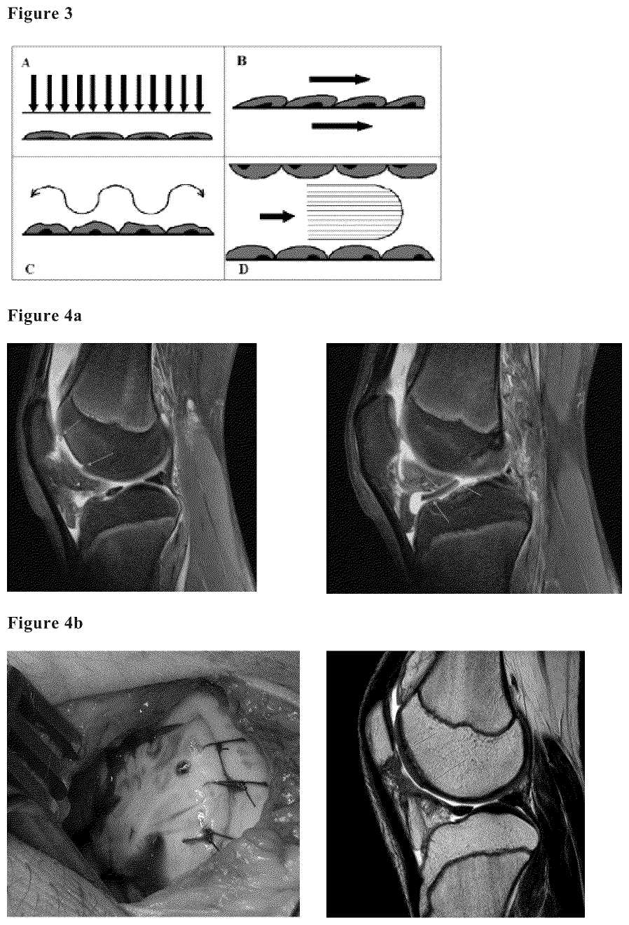

In one embodiment according to the present invention, the mechanical stimulation is compression which can be applied directly to the fused micro construct or directly to the surrounding fluid as hydrostatic pressure (FIG. 3A).

In another embodiment according to the present invention, the mechanical stimulation is tension which can be applied biaxially and/or uniaxially resulting in a temporary structural deformation of the fused micro constructs (FIG. 3B).

In another embodiment according to the present invention, the mechanical stimulation is oscillatory or vibrational stimulation which can be applied to the fused (cartilage) micro construct or directly to the surrounding medium (FIG. 3C).

In another embodiment according to the present invention, the mechanical stimulation is laminar shear stress which can be applied through fluid flow, often to the interior of a fused (cartilage) micro construct lumen (FIG. 3D).

In another embodiment according to the present invention, the mechanical stimulation is a combination of two or more forces selected from the group consisting of compression, tension, oscillatory, vibrational or laminar shear stress.

In one embodiment according to the present invention, the mechanical stimulation will have a duration of between 1 and 15 days, such as between 1 and 12 days, between 1 and 10 days, between 2 and 10 days or between 3 and 10 days.

In one embodiment according to the present invention, the amount of time effective for allowing formation of a cohesive tissue construct, such as a cohesive cartilage construct, is in the range 1-15 days, preferably in the range 1-12 days, more preferably in the range 1-10 days such as in the range 2-10 days or in the range 3-10 days.

The oxygen requirements for optimal in-vitro development of cohesive tissue construct, such as a cohesive cartilage construct, should in theory get close to the hypoxic conditions encountered in native tissue. Thus, in one embodiment according to the present invention, the fused (cartilage) micro construct(s) obtained in step b) are subjected to mechanical stimulation in a hypoxic environment.

Hypoxia refers to low oxygen conditions. About 20.9% of the gas in the atmosphere at 1 atm is molecular oxygen (the partial pressure of oxygen in the atmosphere is 20.9% of the total barometric pressure). If a sample is subjected to a hypoxic environment, the partial pressure of oxygen in the surrounding air is typically less than 20.9%.

When the fused (cartilage) micro construct(s) obtained in step b) are subjected to hypoxic environment, the fused (cartilage) micro construct(s) are typically cultivated in a culture media that is exposed to air where the partial pressure of molecular oxygen (O₂) is less than 20.9%. One way of obtaining such conditions is to incubate the (cartilage) micro constructs in an incubator where the percentage of molecular oxygen inside the incubator is decreased as compared to the percentage of molecular oxygen outside the incubator at 1 atm.

In one embodiment, the hypoxic environment is an environment with a partial pressure of molecular oxygen less than 20%, such as less than 10%, less than 5%, less than 4%, less than 3% or less than 2.5%. In a preferred embodiment, the hypoxic environment is an environment with a partial pressure of molecular oxygen in the range 1 to 10%, such as in the range 1 to 8%, 1 to 6%, 1 to 4% or 2 to 4%. In a particularly preferred embodiment, the hypoxic environment is an environment with a partial pressure of molecular oxygen in the range 2 to 5%, such as in the range 2 to 4%.

The fused (cartilage) micro construct(s) obtained in step b) are typically submerged in a culture medium. By keeping the fused (cartilage) micro construct(s) in a hypoxic environment, the amount of molecular oxygen that is dissolved in the culturing medium will also decrease over time.

The term “hypoxic environment” as used herein refers to the environment surrounding the cell culture media, i.e. it does not refer to the cell culture media per se but refers to the air which is surrounding the cell culture media. Thus, in a hypoxic environment it will take some time before the hypoxic environment will affect the amount of molecular oxygen that is dissolved in the cell culture media, i.e. that it will take some time to establish a new equilibrium.

Thus, in order to reduce the time to establish a new equilibrium, the culture medium may be subjected to treatment to reduce the amount of dissolved molecular oxygen before use. Said in other words, that the fused (cartilage) micro construct(s) obtained in step b) are subjected to mechanical stimulation in a culture media having an amount of dissolved molecular oxygen less than 100% air saturation. If you combine this with keeping the fused (cartilage) micro construct(s) in a hypoxic environment, the fused (cartilage) micro construct(s) will experience a quick and prolonged drop in the amount of molecular oxygen dissolved in said culture media.

Thus, in one embodiment according to the present invention the fused (cartilage) micro construct(s) obtained in step b) is subjected to mechanical stimulation in a culture media wherein the amount of dissolved O₂ in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation. In another embodiment, the amount of dissolved molecular oxygen in said cell culture media is in the range 1-30% air saturation, such as in the range 1-20%, in the range 1-10%, in the range 1-5% or in the range 2-3%.

Percentage air saturation represents a value that is directly linked to the characteristics of the air. In order to transform this value into an absolute value it is necessary to establish a reference point.

The term “culture media at 100% air saturation” as used herein refers to a culture media with an amount of dissolved molecular oxygen which corresponds to a culture media which has been left to stand in air at 1 atm at room temperature (about 20° C.) for sufficient time to establish an equilibrium, i.e. that the molecular oxygen is dissolved at full saturation. Such a culture media is typically prepared by providing culture media and let it stand on a lab bench for enough time to establish an equilibrium. A culture media where the amount of dissolved O₂ is at 80% air saturation is a culture media that has 20% less dissolved molecular oxygen as compared to a culture media at 100% air saturation at 1 atm and room temperature.

It is an object of the invention to produce a transplantable cohesive cartilage construct which has mechanical properties consistent with those of existing cartilage both in terms of compressive strength and lack of friction.

Thus, in one embodiment according to the present invention the fused (cartilage) micro construct(s) obtained in step b) are subjected to mechanical stimulation to allow formation of a cohesive tissue construct, such as a cohesive cartilage construct which contain at least 40% by volume of extracellular matrix, such as at least 60% by volume of extracellular matrix, at least 80% by volume of extracellular matrix, at least 90% by volume of extracellular matrix such as about 95% by volume of extracellular matrix.

### GENERAL ASPECTS

As previously mentioned, several laboratory approaches to production of cartilage tissue in-vitro have been proposed. These generally involve seeding of cultured cells (either chondrocytes or pluripotential stem cells) into a biological or synthetic scaffold. As shown herein, the inventor of the present invention has been able to produce cohesive cartilage construct(s) in-vitro without using any supporting structures such as biological, synthetic or artificial scaffolds.

Thus, in one embodiment according to the present invention, the method of the present invention does not involve use of any supporting materials. Examples of supporting materials are biological, synthetic and artificial scaffolds.

The person skilled in the art will appreciate that step a) must not necessarily be directly followed by step b); and step b) must not necessarily be directly followed by step c). It may e.g. be that the (cartilage) micro construct(s) obtained in step a) are subjected to some kind of treatment before the (cartilage) micro construct(s) are:
subjected to gentle movement; or
put in motion to facilitate contact between the cartilage micro constructs;
and it may also be that the fused (cartilage) micro construct(s) obtained in step b) is subjected to some kind of treatment before the fused (cartilage) micro construct(s) is subjected to mechanical stimulation.

In one embodiment according to the present invention, step b) and step c) are combined into a one step process. Said in other words, that the (cartilage) micro construct(s) obtained in step a) is subjected to gentle movement or put in motion to facilitate contact between the cartilage micro constructs; and subjected to mechanical stimulation to allow formation of a cohesive tissue construct, such as a cohesive cartilage construct.

However, without being bound by theory, it is believed that the more intense the mechanical stimulation is the more important it is that the (cartilage) micro construct(s) have fused into a fused (cartilage) micro construct before being subjected to mechanical stimulation. However, at less intense mechanical stimulation it may be possible that step b) and step c) are performed in a one-step process.

A second aspect and a second alternative aspect of the present invention relates to a cohesive tissue construct, such as a cohesive cartilage construct, produced by the method according to the first aspect and the first alternative aspect respectively of the present invention.

In one embodiment according to the alternative aspect of the present invention, the cohesive tissue construct is a cohesive cartilage construct.

As previously mentioned, several laboratory approaches to production of cartilage tissue in-vitro have been proposed. These generally involve seeding of cultured cells (either chondrocytes or pluripotential stem cells) into a biological or synthetic scaffold. As shown herein, the inventor of the present invention has been able to produce cohesive cartilage tissue in-vitro without using any supporting structures such as biological, synthetic or artificial scaffolds.

Thus, in one embodiment according to the present invention, the cohesive tissue construct, such as cohesive cartilage construct, does not comprise any supporting materials. Examples of supporting materials are biological, synthetic or artificial scaffolds.

As previously disclosed, if the number of cells per drop during hanging-drop cultivation is too high, the spontaneous cell assembling may be hampered, and the resulting structures may become less solid and little consistent. Thus, according to one embodiment of the present invention, the cohesive tissue construct, such as cohesive cartilage construct, is a substantially homogeneous cohesive tissue construct, such as cohesive cartilage construct.

As previously described, nearly 95 percent of articular cartilage is extracellular matrix (ECM) that is produced and maintained by the chondrocytes dispersed throughout it.

Thus, in one embodiment according to the present invention the extracellular matrix of the cohesive tissue construct, such as cohesive cartilage construct, is produced by the cells of the cohesive tissue construct, such as cohesive cartilage construct.

Preferably, all cells of the cohesive tissue construct, such as cohesive cartilage construct, are derived from the cells referred to in the first step of the first aspect or alternative aspect of the present invention.

In another embodiment according to the present invention, the cohesive tissue construct, such as cohesive cartilage construct, contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

In another embodiment according to the present invention, the cohesive tissue construct, such as cohesive cartilage construct, has a base area of at least 5 mm² and a height of at least 2 mm, more preferably the cohesive tissue construct, e.g. cohesive cartilage construct, has a base area of at least 10 mm² and a height of at least 2 mm.

The cohesive tissue construct produced by the method according to the alternative aspect of the present invention and the cohesive cartilage construct produced by the method according to the first aspect of the present invention are suitable for use in surgical methods for repairing damaged tissue in a subject. The result of a successful surgical procedure for repairing damaged tissue in a subject is illustrated in FIGS. 4a and 4b.

Thus, a third aspect of the present invention relates to the cohesive cartilage construct according to the second aspect of the present invention, for use in a surgical method for repairing damaged cartilage in a subject; the surgical method comprising the following step(s): replacing the damaged cartilage in the subject by removing the damaged cartilage and transplanting the cohesive cartilage construct into the site in need for repair.

A third alternative aspect of the present invention relates to the cohesive tissue construct according to the second alternative aspect of the present invention, for use in a surgical method for repairing damaged tissue in a subject; the surgical method comprising the following step(s): replacing the damaged tissue in the subject by removing the damaged tissue and transplanting the cohesive tissue construct into the site in need for repair.

“Transplantation” as used herein refers to a medical procedure in which in-vitro produced construct is placed in the body of a recipient to replace damaged tissue. Tissue that is derived from the recipient of the tissue, i.e. that the subject from which the cells of the cohesive tissue construct or the cohesive cartilage construct is derived is the subject into which the cohesive tissue construct or cohesive cartilage construct is transplanted, is herein referred to as an autograft.

In order to avoid that the transplant is rejected, it is preferred that the subject from which the cells of the cohesive tissue construct or cohesive cartilage construct is derived is the subject into which the cohesive tissue construct or cohesive cartilage construct is transplanted. Thus, it is preferred that the cohesive tissue construct or cohesive cartilage construct is an autograft.

In one embodiment according to the present invention, the cohesive tissue construct is cohesive cartilage construct.

In one embodiment according to the third aspect and third alternative aspect of the present invention, the damaged cartilage is damaged articular cartilage.

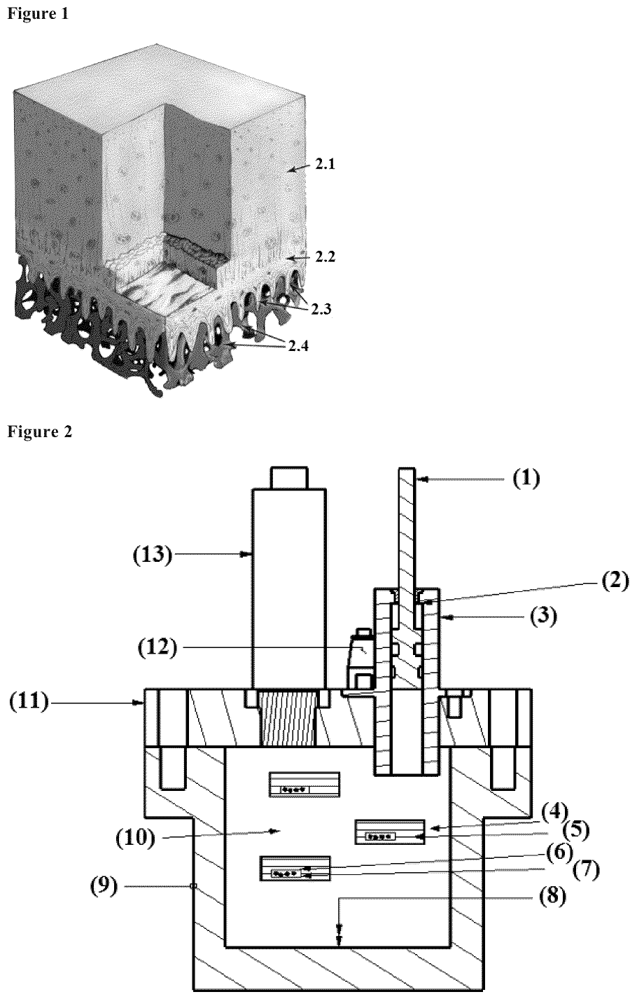

The success of transplantation of a cohesive cartilage construct is critically dependent upon the construct's ability to attach to the site in need for repair. In order to improve the construct's ability to attach to the site in need for repair, the subchondral bone (see FIG. 1) at the site in need for repair may be penetrated or scratched, preferably scratched, to create a minor bleeding from blood vessels on top of the subchondral bone. The stimulated bleeding from the bone will act as a glue and strengthen the attachment of the cohesive cartilage construct to the site in need for repair.

Thus, in one embodiment according to the present invention, subchondral bone (FIG. 1) at the site of the damaged cartilage is penetrated or scratched, preferably scratched, to create a bleeding, preferably a minor bleeding, from blood vessels on top of the subchondral bone; the subchondral bone (FIG. 1) being penetrated or scratched after damaged cartilage has been removed but prior to transplanting the cohesive cartilage construct.

In another embodiment according to the third aspect and third alternative aspect of the present invention, the transplanted cohesive cartilage construct is sutured to surrounding cartilage and/or bone.

In one embodiment according to the third aspect and third alternative aspect of the present invention, the cause of the damaged cartilage is a degenerative disease such as osteoarthritis.

In one embodiment according to the third aspect and third alternative aspect of the present invention, the subject is a human or a non-human animal, preferably a human.

In one embodiment according to the third alternative aspect of the present invention, the damaged tissue is a defect tissue such as defect cartilage.

### EXAMPLES

### Example 1: Isolation and Propagation of Human Articular Chondrocytes (Cell Transplantation 2008; 17:987-996)

Human articular chondrocytes obtained from surplus cells from patients undergoing autologous chondrocyte transplantation were used. Initial biopsies (weight, 300 to 500 mg) were obtained through an arthroscope from non-weight-bearing areas where macroscopically normal cartilage could be obtained. Cartilage biopsies were kept in 0.9% NaCl for approximately 2 hours, and then cut in 1-1.5 mm³ pieces. They were kept for 18 hours in 2-5 ml DMEM/Ham's F-12 (Cat. No. T 481-50, BioChrom Labs, Terre Haute, IN) containing collagenase (Cat. No. C-9407, Sigma Aldrich, Norway AS, Oslo, Norway) at a final concentration of 0.8 mg/ml. The enzyme solution was removed after centrifugation at 200×g and by consecutive washing steps with DMEM/Ham's F-12. Thereafter, the pellet was resuspended in fresh growth medium (DMEM/Ham's F-12 supplemented with 10% bovine calf serum). Cultures were further expanded by trypsinization (Catl. No. T-3924, Sigma), and after repeated washing, resuspended in DMEM/Ham's F-12 supplemented with 10% bovine calf serum.

### Example 2: Hanging-Drop Cultivation of Human Articular Chondrocytes (Cell Transplantation 2008; 17:987-996)

Freshly isolated articular chondrocytes obtained in example 1 were expanded for 3-5 weeks in standard growth medium (DMEM/Ham's F-12 supplemented with 10% bovine calf serum). Monolayers of the articular chondrocytes were dissociated by trypsination and the cell number determined on a hemacytometer. The cell suspension was used to initiate hanging-drop cultures as previously described by Biotechnol. Bioeng. 83:173-180; 2003. Drops of 40 μl containing about 20 000 chondrocytes were dispensed into each well of a 48-well lid (Nunc) and the lid was inverted (day 0). The hanging-drops were exposed to a hypoxic environment equivalent to 3% O₂ during a 6-day period to allow formation of one cartilage micro construct per well.

### Example 3: Gentle Movement and a Low-Oxygen Environment

The cartilage micro constructs obtained in example 2 were transferred to separate non-binding dishes. 8 dishes were filled with 10 cartilage micro constructs each, 8 dishes were filled with 20 cartilage micro constructs each and 4 dishes were filled with 47 cartilage micro constructs each.

All of the dishes were kept for three days in an incubator (HERA cell VIOS 160i-CO2 incubator) with hypoxic (3% oxygen) environment. Half of the dishes (4 dishes filled with 10 cartilage micro constructs each, 4 dishes filled with 20 cartilage micro constructs each, and 2 dishes with 47 cartilage micro constructs each) were allowed to remain unmoved in the hypoxic environment. The other half of the dishes were subjected to gentle movement 3 hours each day by placing the dishes on a mini rocker shaker (PMR-30; Grant-bio, around 10 degrees slope at start, 60 seconds from start of the movement until a full turn had been obtained) in a hypoxic (3% oxygen) environment. Except from the 3 hours of gentle movement, the dishes were kept unmoved for the rest of the day.

FIG. 5a illustrates the results obtained by incubating 20 cartilage micro constructs in an incubator (HERA cell VIOS 160i-CO2 incubator) with hypoxic (3% oxygen) environment for about 24 hours. FIG. 5a shows that the micro constructs remain separated and have not self-assembled into one fused cartilage micro construct. Similar results were also observed for the dishes with 10 and 47 cartilage micro constructs.

FIG. 5b illustrates the results obtained by incubating 20 cartilage micro constructs in an incubator (HERA cell VIOS 160i-CO2 incubator) with hypoxic (3% oxygen) environment for about 24 hours. During those 24 hours, the cartilage micro constructs were subjected to gentle movement for 3 hours. As may be seen from FIG. 5b, the micro constructs have self-assembled into one fused micro construct. Similar results were also observed for the dishes with 10 and 47 cartilage micro constructs.

### Example 4: Hydrodynamic Stimulation

The fused micro construct obtained in example 3 (subjected to gentle movement+hypoxic environment) was transferred to a first container (6). The first container (6) has an inner base area of about 1 cm² and a height of 2 mm. The floor and the ceiling of the first container (6), each having a surface area of about 1 cm², are semipermeable membranes (Durapore PVDF, Merck Life Science A/S, Norway, SVLP 04700, pore size 5 μm). The first container (6) being filled with growth medium (DMEM/Ham's F-12 supplemented with 10% bovine calf serum) which has been exposed to low oxygen environment (3%) for 2 hours prior to being filled into the first container (6).

The first container (6) is then placed inside a second container (4). The second container (4) has a base area of about 30 cm² and a height of 10 mm. The ceiling of the second container (4) with a surface area of about 30 cm² is an impermeable membrane (Silicone membrane 40 Shore A, thickness 1 millimeter, TeknoLab A/S, Ski, Norway). The second container (4) is filled with growth medium (DMEM/Ham's F-12 supplemented with 10% bovine calf serum) which has been exposed to low oxygen environment (3%) for 2 hours prior to being filled into the second container (4).

The second container (4) is then placed inside a bioreactor-chamber (9). The bioreactor-chamber (9) has the shape of a circular cylinder. All faces of the chamber (9) are of a non-flexible material and the chamber (9) is filled with distilled water (10). The distilled water (10) being in fluid communication with a piston (1). The piston (1) being connected to a pneumatic driven motor which is able to pull/push the piston (1) and thereby create a change in pressure within the bioreactor-chamber (9) and indirectly a change in pressure within the second (4) and the first chambers (6) respectively.

A force of 1.4 MPa is applied to the piston (1) for 1 second followed by a pressure release for 1 second. This hydrodynamic stimulation, in the form of a fluctuating pressure, was continued for one hour daily over a period of two days.

The force applied to the piston (1) results in an increased pressure within the bioreactor-chamber (9) which indirectly will also change the pressure within the second (4) and first containers (6). When there is no hydrodynamic stimulation, the first container (6) is incubated in a low oxygen environment (3%).

FIG. 7 illustrates the result obtained by hydrodynamic stimulation. The fused micro constructs have self-assembled into a small cohesive tissue construct (indicated by the black arrow) measuring about 6 mm in length and 2 mm in breadth.

The small cohesive tissue construct was moved out of the first container (6) and into formalin. Histology of the tissue is shown in FIG. 8 which clearly shows that the chondrocytes have created small cohesive tissue constructs indicating a transition from cell culture to a bona fide tissue. Moreover, some of the chondrocytes are already lying in the lacuna-like spaces (thin arrows), and there is focally seen forming of basophilic acid glycosaminoglycan-rich extracellular matrix (thick arrows) imparting the newly formed tissue resemblance to an immature cartilage.

1. A method for in-vitro production of a cohesive cartilage construct, the method comprising the following steps:
a) propagating chondrogenic cells derived from a subject to allow formation of one or more cartilage micro constructs;
b) putting a plurality of the cartilage micro constructs in motion to facilitate contact between the cartilage micro constructs and thereby allow formation of one or more fused cartilage micro constructs; and
c) subjecting one or more of the fused cartilage micro constructs to mechanical stimulation in a hypoxic environment to allow formation of a cohesive cartilage construct.

2. The method according to claim 1, wherein
the cells in step a) are propagated in a hypoxic environment; and/or
the plurality of cartilage micro constructs in step b) are put in motion to facilitate contact between the cartilage micro constructs in a hypoxic environment.

3. The method according to claim 1, wherein the hypoxic environment is an environment with a partial pressure of molecular oxygen (O2) less than 20%, such as less than 18%, less than 16%, less than 14%, less than 12%, less than 10%, less than 8%, less than 6%, less than 5%, less than 4%, less than 3% or less than 2.5%.

4. The method according to claim 1, wherein the hypoxic environment is an environment with a partial pressure of molecular oxygen (O2) less than 10%.

5. The method according to claim 1, wherein the chondrogenic cells in step a) are propagated by a technique, such as hanging drop cultivation, suitable to allow formation of one or more three-dimensional cell structures.

6. The method according to claim 1, wherein the one or more cartilage micro constructs referred to in step a) are three-dimensional cell structures, such as spheroids.

7. The method according to claim 1, wherein the plurality of cartilage micro constructs in step b) are put in circular motion, such as uniform circular motion or smooth uniform circular motion, to facilitate contact between the cartilage micro constructs.

8. The method according to claim 1, wherein the plurality of cartilage micro constructs in step b) are put in motion by subjecting the plurality of cartilage micro constructs in step b) to tilting about one axis to facilitate contact between the cartilage micro constructs.

9. The method according to claim 8, wherein tilting about one axis is achieved using a shaker, such as a rocker shaker or a mini rocker shaker.

10. The method according to claim 1, wherein the plurality of cartilage micro constructs in step b) are put in motion by subjecting the plurality of cartilage micro constructs in step b) to tilting about two or more axis, such as more than two independent horizontal axes, to facilitate contact between the cartilage micro constructs.

11. The method according to claim 1, wherein the chondrogenic cells are chondrocytes.

12. The method according to claim 1, wherein the subject is human or non-human, preferably human.

13. The method according to claim 1, wherein
the chondrogenic cells in step a) and/or the plurality of the cartilage micro constructs in step b) and/or the one or more fused cartilage micro constructs in step c) are submerged in a cell culture media; and
the amount of dissolved molecular oxygen in said cell culture media is less than 100% air saturation, more preferably less than 80% air saturation, and even more preferably less than 60% air saturation and most preferably less than 40% air saturation, such as less than 40% air saturation, less than 30% air saturation, less than 20% air saturation or less than 10% air saturation.

14. The method according to claim 1, wherein the mechanical stimulation is selected from the group consisting of compression, tension, oscillatory and/or vibrational stimulation, shear stress and any combination thereof.

15. The method according to claim 14, wherein
the compression is applied directly to the fused cartilage micro constructs; and/or
the one or more fused cartilage micro constructs in step c) are submerged in a cell culture media and the compression is applied to the surrounding cell culture media.

16. The method according to claim 14, wherein tension is applied biaxially and/or uniaxially resulting in a temporary structural deformation of the fused cartilage micro constructs.

17. The method according to claim 14, wherein
oscillatory and/or vibrational stimulation is applied directly to the fused cartilage micro constructs; and/or
the one or more fused cartilage micro constructs in step c) are submerged in a cell culture media and the oscillatory and/or vibrational stimulation is applied to the surrounding cell culture media.

18. The method according to claim 1, wherein the mechanical stimulation is hydrodynamic stimulation.

19. The method according to claim 1, wherein the one or more fused cartilage micro constructs in step c) are submerged in a cell culture media and the mechanical stimulation is hydrodynamic stimulation.

20. The method according to claim 1, wherein the mechanical stimulation does not involve exposing the fused cartilage micro constructs and/or cohesive cartilage construct to a pressure ≥12 MPa.

21. The method according to claim 1, wherein the mechanical stimulation does not involve exposing the fused cartilage micro constructs and/or cohesive cartilage construct to a pressure >10 MPa.

22. The method according to claim 1, wherein the mechanical stimulation does not involve exposing the fused cartilage micro constructs and/or cohesive cartilage construct to a pressure ≥8 MPa.

23. The method according to claim 1, wherein the mechanical stimulation does not involve exposing the fused cartilage micro constructs and/or cohesive cartilage construct to a pressure ≥7 MPa.

24. The method according to claim 1, wherein the mechanical stimulation does not involve exposing the fused cartilage micro constructs and/or cohesive cartilage construct to a pressure ≥5 MPa.

25. The method according to claim 1, wherein
the hypoxic environment is an environment with a partial pressure of molecular oxygen (O2) less than 10%; and
the chondrogenic cells in step a) are propagated by a technique, such as hanging drop cultivation, suitable to allow formation of one or more three-dimensional cell structures.

26. The method according to claim 1, wherein
the hypoxic environment is an environment with a partial pressure of molecular oxygen (O2) less than 10%; and
the chondrogenic cells in step a) are propagated by a technique, such as hanging drop cultivation, suitable to allow formation of one or more three-dimensional cell structures; and
the one or more cartilage micro constructs referred to in step a) are three-dimensional cell structures, such as spheroids.

27. The method according to claim 1, wherein
the cells in step a) are propagated in a hypoxic environment;
the plurality of cartilage micro constructs in step b) are put in motion to facilitate contact between the cartilage micro constructs in a hypoxic environment;
the hypoxic environment is an environment with a partial pressure of molecular oxygen (O2) less than 10%; and
the chondrogenic cells in step a) are propagated by a technique, such as hanging drop cultivation, suitable to allow formation of one or more three-dimensional cell structures.

28. The method according to claim 1, wherein step b) and step c) are combined into a one step process.

29. The method according to claim 28, wherein a plurality of the cartilage micro constructs obtained in step a) are put in motion to facilitate contact between the cartilage micro constructs and subjected to mechanical stimulation in a hypoxic environment thereby allowing formation of a cohesive cartilage construct.

30. A cohesive cartilage construct produced by the method according to claim 1.

31. The cohesive cartilage construct according to claim 30, wherein the cohesive cartilage construct contains at least 40% by volume of extracellular matrix, such as at least 60% by volume of extracellular matrix, at least 80% by volume of extracellular matrix, at least 90% by volume of extracellular matrix such as about 95% by volume of extracellular matrix.

32. The cohesive cartilage construct according to claim 30, wherein the cohesive cartilage construct has a base area of at least 5 mm² and a height of at least 2 mm.

33. The cohesive cartilage construct according to claim 30 for use in a surgical method for repairing damaged cartilage in a subject; the surgical method comprising the following step(s): replacing the damaged cartilage in the subject by removing the damaged cartilage and transplanting the cohesive cartilage construct.

34. The cohesive cartilage construct for use according to claim 33, wherein the subject from which cells of the cohesive cartilage construct are derived is the subject into which the cohesive cartilage construct is transplanted.

35. The cohesive cartilage construct for use according to claim 33, wherein subchondral bone at the site of the damaged cartilage is
penetrated to create a bleeding from blood vessels on top of the subchondral bone; and/or
scratched to create a minor bleeding from blood vessels on top of the subchondral bone;
after damaged cartilage has been removed but prior to transplanting the cohesive cartilage construct.

36. The cohesive cartilage construct for use according to claim 33, wherein the cause of the damaged cartilage is a degenerative disease, such as osteoarthritis.
